(19) United States
(12) Patent Application Publication
(10) Pub. No.: US 2025/0339262 A1
(43) Pub. Date: Nov. 6, 2025

(54) ARTIFICIAL INTRAOCULAR LENS

(71) Applicant: Brian Rogers, Nashville, TN (US)

(72) Inventor: Brian Rogers, Nashville, TN (US)

(21) Appl. No.: 18/653,423

(22) Filed: May 2, 2024

(51) Int. Cl. A61F 2/16

(52) U.S. Cl. CPC A61F 2/1629; A61F 2/1651; A61F 2230/001; A61F 2230/0013; A61F 2250/0003

(57) ABSTRACT

An intraocular lens for implantation in a capsular bag of an eye comprising a membrane, an internal cavity, and a gas. The membrane may include an anterior face and a posterior face. The anterior and posterior faces may be joined together at a periphery of the membrane. The membrane may include at least one optical element associated therewith. The internal cavity may be defined within the membrane. The gas may be positioned within the internal cavity. The gas may be operable to separate the anterior and posterior faces of the membrane. The intraocular lens may be neutrally buoyant within the capsular bag.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### FIELD OF THE DISCLOSURE

The present disclosure relates generally to artificial intraocular lenses. More particularly, the present invention relates to artificial intraocular lenses configured for implantation in a lens chamber of a user's eye.

### BACKGROUND

Cataracts remain one of the most prevalent ocular diseases in the world and are one of the leading causes of impaired vision. In some extreme cases, cataracts can even lead to blindness. Visual disability from cataracts accounts for more than 8 million physician office visits per year. When the disability from cataracts affects or alters an individual's lifestyle, surgical lens removal with artificial intraocular lens implantation is the preferred method of treating the functional limitations. Cataract surgery remains one of the most common surgical procedures and is widely accepted by the public.

The crystalline lens of the eye is deformable along a range of powers, but its natural elasticity declines as a function of age or other secondary factors. Eventually, the lens becomes so rigid that it forms a cataract. A cataract is any opacity of a patient's lens, whether it is a localized opacity or a diffused general loss of transparency. Once the cataract has become sufficiently dense, it must be removed.

In treating a cataract, the surgeon removes the crystalline lens from the lens capsule and replaces it with an artificial intraocular lens. The typical artificial intraocular lens provides a selected focal length that optically corrects the patient for distance. However, most artificial intraocular lenses do not correct near vision issues, and patients are left needing reading glasses.

Recently, some artificial intraocular lenses have attempted to correct patients for near and far focal points but fail for several reasons. One issue is that these artificial intraocular lenses use translational motion of two high-index optical elements. The distances the pair of high-index optical elements can move away from one another is limited. The greater the separation between the pair of high-index optical elements, the greater the magnification. One problem is that the capsular bag in which the artificial intraocular lens is implanted is finite in space and the ciliary body is too weak to create large dioptric shifts. Moreover, in order to focus the lens for distance, the ciliary body must contract. In other words, in order for the patient to see at a distance, they must contract a muscle. This often leads to overuse of the muscles associated with the eye causing perpetual headaches and blurry vision. Various other issues exist, including that these artificial intraocular lenses attempt to mimic the refractive index of the natural crystalline lens and are too complex for widespread use.

Accordingly, a need exists for improvements in artificial intraocular lenses.

### BRIEF SUMMARY

This Brief Summary is provided to introduce a selection of concepts in a simplified form with respect to those further described below. This Brief Summary is not intended to identify key features or essential features of an invention as disclosed herein, or to otherwise limit the scope of an invention as disclosed herein, unless otherwise specifically noted.

One aspect in accordance with the present disclosure is an intraocular lens for implantation in a capsular bag of an eye. The intraocular lens may comprise a membrane, an internal cavity, and a gas. The membrane may include an anterior face and a posterior face. The anterior and posterior faces may be joined together at a periphery of the membrane. The membrane may include at least one optical element associated therewith. The internal cavity may be defined within the membrane. The gas may be positioned within the internal cavity and operable to separate the anterior and posterior faces of the membrane. The intraocular lens may be neutrally buoyant within the capsular bag.

In accordance with another aspect of the disclosure, the at least one optical element may be convex is shape.

In accordance with another aspect of the disclosure, the gas may be positioned adjacent to the posterior face of the membrane.

In accordance with another aspect of the disclosure, the internal cavity may be plano-convex in shape.

In accordance with another aspect of the disclosure, the at least one optical element may include a first optical element and a second optical element. The at least one optical element may be bi-concave in shape.

In accordance with another aspect of the disclosure, the internal cavity may be positioned between the first and second optical elements.

In accordance with another aspect of the disclosure, the internal cavity may be bi-convex in shape.

In accordance with another aspect of the disclosure, the internal cavity may be substantially flat.

In accordance with another aspect of the disclosure, the membrane may include two or more layers each comprising a different material.

In accordance with another aspect of the disclosure, the membrane may be substantially impervious to the gas positioned within the internal cavity.

In accordance with other aspects of the disclosure, the gas positioned within the internal cavity may be any gas currently approved for medical use.

In accordance with another aspect of the disclosure, a volume of the at least one optical element may be substantially equal to a volume of the gas positioned within the internal cavity.

Another aspects of the present disclosure is an intraocular lens for implantation in a capsular bag of an eye. The intraocular lens may comprise a membrane and an internal cavity. The membrane may include an anterior face and a posterior face. The anterior and posterior faces may be joined together at a periphery of the membrane. The membrane may include at least one optical element associated therewith. The internal cavity may be defined within the membrane. The internal cavity may be selectively configurable in an inflated configuration and a deflated configuration. In the inflated configuration the internal cavity may include a gas positioned therein.

In accordance with another aspect of the disclosure, the at least one optical element may be convex in shape. The internal cavity may be positioned adjacent to the posterior face of the membrane. The internal cavity may be plano-convex in shape.

In accordance with another aspect of the disclosure, the at least one optical element may include a first optical element and a second optical element. The at least one optical element may be bi-concave in shape. The internal cavity may be positioned between the first and second optical elements.

In accordance with another aspect of the disclosure, the internal cavity may be bi-convex in shape.

In accordance with another aspect of the disclosure, the internal cavity may be substantially flat.

In accordance with another aspect of the disclosure, the membrane may include two or more materials positioned therein.

In accordance with another aspect of the disclosure, the two or more materials of the membrane may be layered.

Numerous objects, features and advantages of a system and method as disclosed herein will be readily apparent to those skilled in the art upon a review of the following description in conjunction with the accompanying drawings.

### BRIEF DESCRIPTION OF THE DRAWINGS

FIG. 1 is a schematic side view of an exemplary embodiment of a human eye.

FIG. 2 is a front elevation view of an artificial intraocular lens in accordance with the present disclosure.

FIG. 3 is a side elevation view of an exemplary embodiment of the artificial intraocular lens of FIG. 2 wherein an at least one optical element is convex.

FIG. 4 is a side elevation view of an exemplary embodiment of the artificial intraocular lens of FIG. 2 wherein an at least one optical element is bi-concave and an internal chamber is bi-convex.

FIG. 5 is a side elevation view of an exemplary embodiment of the artificial intraocular lens of FIG. 2 wherein an at least one optical element is bi-concave and an internal chamber is substantially flat.

FIG. 6 is a side elevation view of an exemplary embodiment of the artificial intraocular lens of FIG. 2 wherein an internal chamber is positioned at a bottom portion of a membrane of the lens.

### DETAILED DESCRIPTION

Reference will now be made in detail to embodiments of the present disclosure, one or more drawings of which are set forth herein. Each drawing is provided by way of explanation of the present disclosure and is not a limitation. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

Thus, it is intended that the present disclosure covers such modifications and variations as come within the scope of the appended claims and their equivalents. Other objects, features, and aspects of the present disclosure are disclosed in, or are obvious from, the following detailed description. It is to be understood by one of ordinary skill in the art that the present discussion is a description of exemplary embodiments only and is not intended as limiting the broader aspects of the present disclosure.

The words “connected,” “attached,” “joined,” “mounted,” “fastened,” and the like should be interpreted to mean any manner of joining two objects.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan

A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or multiple components.

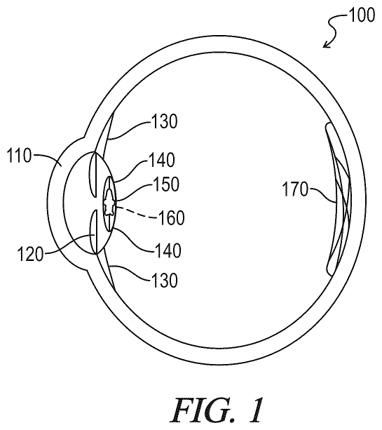

Referring now to the figures, and specifically FIG. 1, a human eye is schematically shown and generally designated by the number 100. The eye 100 includes a cornea 110, pupil 120, ciliary muscles 130, zonular fibers 140, transparent elastic capsular bag 150, crystalline lens 160, and retina 170. Crystalline lens 160 is composed of viscous, gelatinous transparent fibers, arranged in a layered structure, and is disposed in transparent elastic capsular bag 150. The capsular bag 150 may also be referred to herein as a capsule 150. The capsular bag 150 is joined by zonular fibers 140 around its circumference to ciliary muscles 130, which are in turn attached to the inner surface of the eye 100.

The capsular bag 150 and crystalline lens 160 may take on a spherical shape. However, when suspended within the eye 100 by zonular fibers 140, the capsular bag 150 moves between a moderately convex shape (when the ciliary muscles 130 are relaxed) to a highly convex shape (when the ciliary muscles 130 are contracted). When the ciliary muscles 130 relax, capsular bag 150 and crystalline lens 160 are pulled about the circumference into an elliptical shape, thereby flattening the crystalline lens 160 and allowing for far vision. When the ciliary muscles 130 contract, the capsular bag 150 and crystalline lens 160 assume a more spherical shape, thus increasing the diopter power of the lens and allowing for near vision.

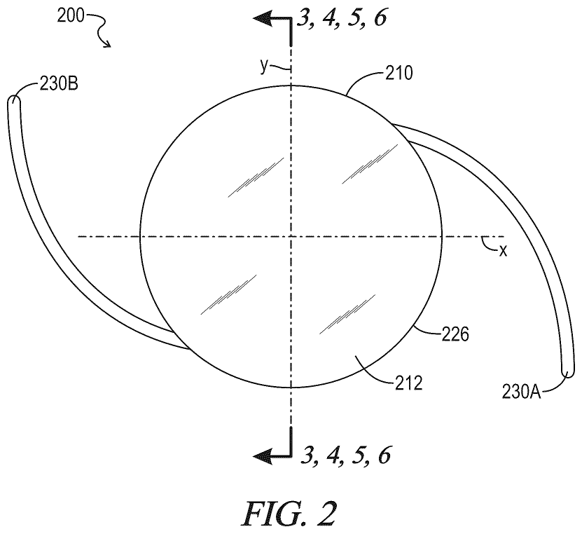

Referring now to FIG. 2, an exemplary embodiment of an artificial intraocular lens is shown and generally designated by the number 200. The artificial intraocular lens 200 may be configured for implantation in the capsular bag 150 of an eye 100. In accordance with certain aspects of the disclosure, the artificial intraocular lens 200 may have a diameter in a range of from 8.5 millimeters to 11.5 millimeters and preferably from 9 millimeters to 11 millimeters. Most preferably, the artificial intraocular lens 200 may have a diameter of 10 millimeters. In accordance with certain aspects of the disclosure, the artificial intraocular lens 200 may have a thickness in a range of from 3 millimeters to 6 millimeters and preferably from 4 millimeters to 5 millimeters. Most preferably, the artificial intraocular lens 200 may have a thickness of 4.5 millimeters. The artificial intraocular lens 200 may substantially fill the capsular bag 150 when positioned within a patient's eye.

The artificial intraocular lens 200 may include a flexible or semi-flexible membrane 210. The membrane 210 may be capable of being deformed and/or rolled such that the artificial intraocular lens 200 can be implanted into the capsular bag 150 of the eye 100 through a small incision. The artificial intraocular lens 200, including the membrane 210, may be made from a material that does not resist deformation or is characterized as having a low Young's modulus.

In accordance with certain aspects of the disclosure, the membrane 210 may include a single material. In accordance with other aspects of the disclosure, the membrane 210 may include two or more materials positioned therein. For example, the membrane 210 may include two or more layers. Each of the two of more layers may be comprised of a different material. In one exemplary aspect, the membrane 210 may include two silicone polymer layers with a gallium-indium eutectic alloy layer disposed between the two silicone polymer layers. In another example, the membrane 210 may include silicon polymer with a carbon nanotube structure associated therewith. The silicon polymer may be impregnated with the carbon nanotube structure. In accordance with certain aspects of the disclosure, the membrane 210 may include a carbon nanostructure. The carbon nanostructure may include carbon nanotubes (single-walled or multi-walled), graphene, and/or carbon nanospheres. The carbon nanostructure may be configured in various patterns and arrangements known in the art.

The membrane 210 may include an anterior face 212 and a posterior face 214. The anterior face 212 may refer to the face of the membrane 210 closest to the retina 170 when the artificial intraocular lens 200 is implanted in the capsular bag 150. The posterior face 214 may refer to the face of the membrane 210 furthest from the retina 170 when the artificial intraocular lens 200 is implanted in the capsular bag 150. The anterior face 212 and the posterior face 214 may be joined together at a periphery 226 of the membrane 210. The periphery 226 may comprise a circumferential edge configured to engage a circumferential region of the capsular bag 150 of the eye 100.

Each of the anterior and posterior faces 212, 214 may comprise a central region and a peripheral region. In accordance with certain aspects of the disclosure, each of the anterior and posterior faces 212, 214 may include a gradient of thickness that increases radially from the peripheral region to the central region. In accordance with other aspects of the disclosure, each of the anterior and posterior faces 212, 214 may include a gradient of thickness that decreases radially from the peripheral region to the central region. These thickness profiles may be operable to prevent bulges or depressions of the anterior and posterior faces 212, 214 of the membrane 210 and to encourage overall stability of the artificial intraocular lens 200.

The membrane 210 may include an internal cavity 220 defined therein. The internal cavity 220 may be configured in a variety of shapes in accordance with certain aspects of the present disclosure. In accordance with certain aspects of the disclosure, the membrane 210 may include a single internal cavity 220. In accordance with other aspects of the disclosure, the membrane 210 may include two or more internal cavities 220.

In accordance with certain aspects of the disclosure, the internal cavity 220 may be selectively configurable in an inflated configuration and a deflated configuration. In the inflated configuration, the internal cavity 220 may include a gas 228 positioned or disposed therein. In the deflated configuration, the internal cavity 220 may be substantially devoid of gas. A patient may still experience distance vision when the artificial interocular lens 200 is in the deflated configuration via an optical element 250. In accordance with other aspects of the disclosure, the internal cavity 220 may exist only in the inflated configuration wherein gas 228 is positioned within the internal cavity 220. In accordance with certain aspects of the disclosure, the membrane 210 may be substantially impervious to gas 228 when the gas 228 is positioned within the internal cavity 220. “Substantially impervious” as used herein may refer to a membrane 210 wherein the gas 228 does not pass through. “Substantially impervious” may also refer to a membrane 210 wherein the gas 228 passes through at a slow rate such that the internal cavity 220 deflates over an extended period of time.

In accordance with certain aspects of the disclosure, the gas 228 may substantially fill the internal cavity 220. The gas 228 may be operable to separate the anterior and posterior faces 212, 214 of the membrane 210. In accordance with certain aspects of the disclosure, the gas 228 may be a large molecule gas, such as xenon or sulfur hexafluoride to name a few examples. One advantage of the gas 228 may be that it contributes to the overall flexibility of the artificial intraocular lens 200. In certain optional embodiments, the gas 228 may provide accommodating power to the artificial intraocular lens 200. Accordingly, the gas 228 may provide a range of accommodation of up to at least 5 diopters, preferably up to at least 10 diopters, and most preferably up to at least 15 diopters. The gas 228 may have a relatively low index of refraction, such as in a range of from 1.0 to 1.5, and preferably in a range of from 1.0 to 1.1. In accordance with other aspects of the present disclosure, a fluid or gel having a low index of refraction may be used in place of the gas 228.

In accordance with certain aspects of this disclosure, the artificial intraocular lens 200 may include at least one optical element 250 associated with the membrane 210. The at least one optical element 250 may be positioned within the membrane 210 or may form a portion of the membrane 210. In accordance with certain aspects of this disclosure, each of the at least one optical element 250 may be coupled to or form a portion of the anterior face 212 and/or the posterior face 214 of the membrane 210. The at least one optical element 250 may be flexible in nature similar to the crystalline lens 160. In accordance with other aspects of the disclosure, the at least one optical element 250 may be rigid or semi-rigid. The at least one optical element 250 may include variable or static curvature and may exist in a variety of shapes. The at least one optical element may be convex, plano convex, biconvex, plano concave, or meniscus-shaped in accordance with certain aspects of the disclosure.

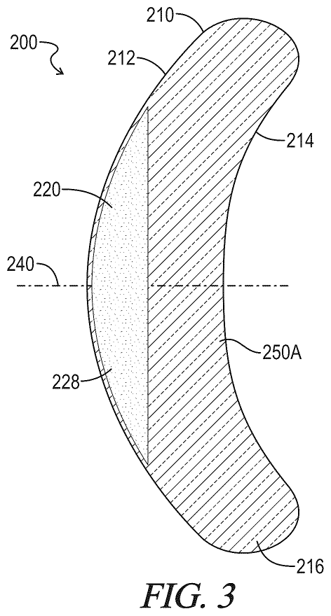

In accordance with certain aspects of the disclosure, as shown in FIG. 3, the at least one optical element 250 may include a single optical element 250A. In such exemplary embodiments, the at least one optical element 250 may have a convex shape. An anterior face of the at least one optical element 250 may be curved and a posterior face of the at least one optical element 250 may be substantially flat. The at least one optical element 250 may be positioned adjacent to the anterior face 212 of the membrane 210. The internal cavity 220 may be positioned adjacent to the posterior face 214 of the membrane 210. The internal cavity 220 may be plano-convex in shape. Thus, an anterior side of the internal cavity 220 may be flat and a posterior side of the internal cavity 220 may be curved.

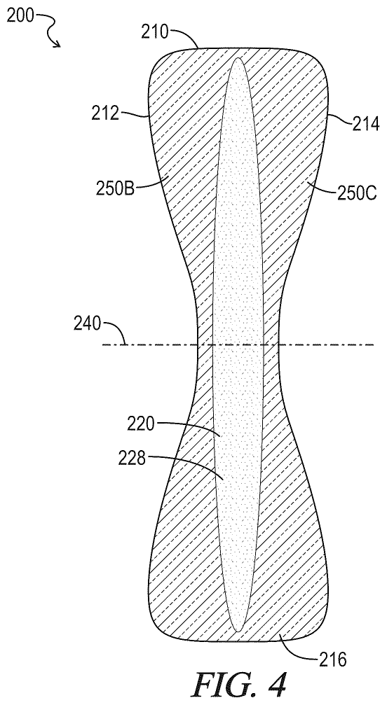

In accordance with certain aspects of the disclosure, as shown in FIG. 4, the at least one optical element 250 may include a first optical element 250B and a second optical element 250C. The first optical element 250B may be coupled to the posterior face 214 of the membrane 210. The second optical element 250C may be coupled to the anterior face 212 of the membrane 210. The at least one optical element 250 may be bi-concave in shape. The internal cavity 220 may be positioned between the first and second optical elements 250B, 250C. The internal cavity 220 may be bi-convex in shape.

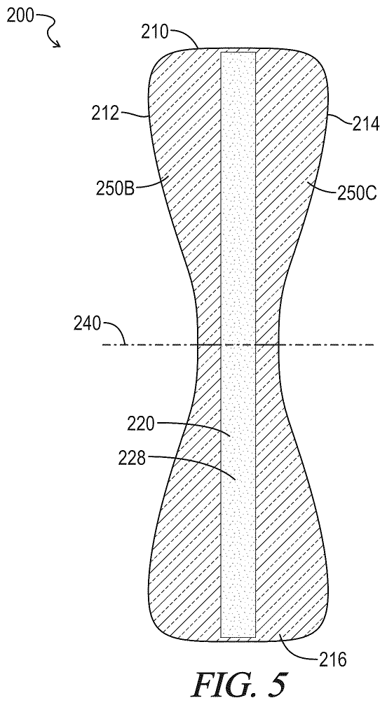

In accordance with certain aspects of the disclosure, as shown in FIG. 5, the at least one optical element 250 may include first and second optical elements 250B, 250C. The first optical element 250B may be coupled to the posterior face 214 of the membrane 210. The second optical element 250C may be coupled to the anterior face 212 of the membrane 210. The at least one optical element 250 may be bi-concave in shape. The internal cavity 220 may be positioned between the first and second optical elements 250B, 250C. The internal cavity 220 may be substantially flat.

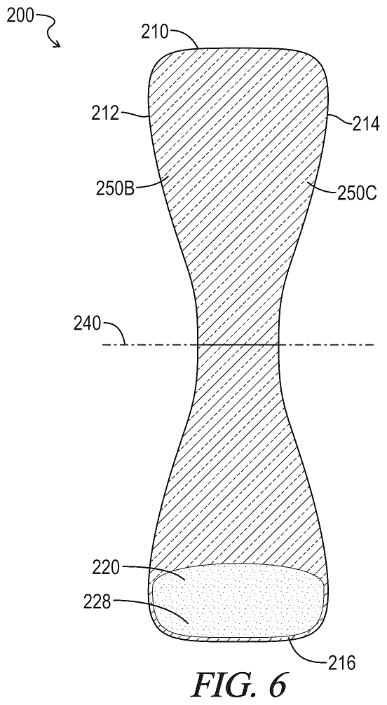

In accordance with certain aspects of the disclosure, as shown in FIG. 6, the internal cavity 220 may be substantially round. The bottom portion 216 of the membrane 210 may be weighted. One such advantage of the weighted bottom portion 216 may be that the artificial intraocular lens 200 is able to maintain a desired orientation within the capsular bag 150.

While certain exemplary embodiments are shown in FIGS. 3-6, it is within the spirit and scope of the present disclosure for the proportions and/or size of certain elements of the artificial intraocular lens 200 to vary. For example, the internal cavity 220, at least one optical element 250, and/or other elements of the artificial intraocular lens 200 may be sized differently relative one another.

The membrane 210, along with all other portions of the artificial intraocular lens 200, may be transparent or at least semi-transparent. Thus, the membrane 210 may be configured to allow light to pass through such that it reaches the retina 170 of the eye 100. The artificial intraocular lens 200 may be neutrally buoyant such that the artificial intraocular lens 200 neither floats nor sinks in the aqueous humor that may be found within the capsular bag 150. A volume of the at least one optical element 250 may be substantially equal to a volume of the gas 228 positioned within the internal cavity 220. One advantage of the artificial intraocular lens 200 being neutrally buoyant may be the increased overall stability of the artificial intraocular lens 200.

The artificial intraocular lens 200 may include at least one curved haptic 230 extending from the membrane 210. More specifically, each of the at least one haptic 230 may be coupled to the periphery 226 of the membrane 210. Each of the at least one haptic 230 may be made of a resilient elastic material that permits the haptic 230 to partially resist forces applied to the haptic 230. In accordance with certain aspects of the disclosure, the at least one may include a first haptic 230A and a second haptic 230B. The first haptic 230A may preferably be located generally 180 degrees from the second haptic 230A.

When the artificial intraocular lens 200 is placed in the capsular bag 150 of an eye 100, the at least one haptic 230 may be operable to resist movement of the artificial intraocular lens 200 relative to the capsular bag 150. More specifically, the at least one haptic 230 may be operable to resist translation of the artificial intraocular lens 200 relative to the capsular bag 150 in the X-Y plane, as defined in FIG. 1, and/or to resist rotation of the entire artificial intraocular lens 200 relative to the capsular bag 150 about a central axis 240 of the artificial intraocular lens 200. The surfaces of each of the at least one haptic 230 may press against the walls of the capsular bag 150 to stabilize the artificial intraocular lens 200 within the capsular bag 150. The at least one haptic 230 may also be operable to center the artificial intraocular lens 200 within the capsular bag 150.

The artificial intraocular lens 200, while stabilized relative to the capsular bag 150, may still move with or relative to the capsular bag 150 and thus be accommodating. Accordingly, when the ciliary muscles 130 relax, the capsular bag 150 and the artificial intraocular lens 200 may be pulled about the periphery 226 of the membrane 210 into an elliptical shape, thereby flattening the artificial intraocular lens 200 to allow for far vision. When the ciliary muscles 130 contract, the capsular bag 150 and the artificial intraocular lens 200 may assume a more spherical shape, thus increasing the diopter power of the artificial intraocular lens 200 and allowing for near vision.

Various features of the artificial intraocular lens 200 may be intended to maximize the extent to which radially compressive forces are transmitted to the internal cavity 220 and the at least one optical element 250. Moreover, the artificial intraocular lens 200 may include similar flexibility to a crystalline lens 160. One advantage of at least these features may be that the artificial intraocular lens 200 provides a large range of accommodation. A patient may enjoy near and far vision without the need for corrective glasses.

The gas 228 positioned within the internal cavity 220 may be operable as an optical element. Small changes to the shape of position of the artificial intraocular lens 200, and more specifically the gas 228 positioned in the internal cavity 220, may produce changes to the power of the artificial intraocular lens 200.

In accordance with certain aspects of the disclosure, the membrane 210 of the artificial intraocular lens 200 may include a valve. The valve may be operably connected to the internal cavity 220. In accordance with certain aspects of the disclosure, the valve may be selectively moveable between a closed position and an open position. In the closed position, the internal cavity 220 may be sealed. In the open position, the valve may be configured to allow for the insertion of gas 228 or other material. One advantage of the present disclosure may be that adjustments can be made to the internal cavity 220 (i.e., volume of gas therein) via the valve after and/or before the artificial intraocular lens 200 has been inserted into the capsular bag 150. In accordance with certain aspects of the disclosure, the valve may be in an open position upon initial injection of gas 228 and permanently moved to the closed position thereafter.

Another aspect of the present disclosure may be a method of implanting the artificial intraocular lens 200 into the capsular bag 150 of the eye 100. The method may include creating an incision in the eye 100. More specifically, the incision may be made in the cornea 110 of the eye 100 such that the capsular bag 150 may be accessed. In accordance with certain aspects of the disclosure, the incision may be made via a laser or like device.

The method may further include removing the crystalline lens 160. Removing the crystalline lens 160 may include breaking up the crystalline lens 160 into two or more pieces via ultrasound waves. The two or more pieces of the crystalline lens 160 may be removed from the capsular bag 150 via suction.

The method may further include preparing the artificial intraocular lens 200 for implantation. Preparing the artificial intraocular lens 200 may include inserting gas 228 into the internal cavity 220 via the valve and closing the valve such that the internal cavity 220 is substantially sealed.

The method may further include inserting the artificial intraocular lens 200 into the capsular bag 150. The artificial intraocular lens 200 may be rolled, folded, or otherwise manipulated such that it may be inserted through the incision.

In certain instances, a patient may have had a previous cataract surgery. Thus, the crystalline lens 160 may have been removed from the capsular bag 150 and an artificial lens implanted into the capsular bag 150. The preexisting artificial lens may be non-accommodating. Thus, the patient may only enjoy far vision. Another aspect of the present disclosure may be a method of implanting the artificial intraocular lens 200 into the capsular bag 150 that contains the preexisting artificial lens.

The method may include creating an incision in the eye 100. More specifically, the incision may be made in the cornea 110 of the eye 100 such that the capsular bag 150 may be accessed.

The method may further include preparing the artificial intraocular lens 200 for implantation. Preparing the artificial intraocular lens 200 may include inserting gas 228 into the internal cavity 220 via the valve and closing the valve such that the internal cavity 220 is sealed.

The method may further include inserting the artificial intraocular lens 200 into the capsular bag 150. The artificial intraocular lens 200 may be rolled, folded, or otherwise manipulated such that it may be inserted through the incision. The artificial intraocular lens 200 may be arranged in relation to the preexisting artificial lens such that the patient may enjoy near vision as well as far vision. The preexisting artificial lens may be operable as a scaffold for the artificial intraocular lens 200.

Thus, it is seen that the apparatus and methods of the present disclosure readily achieve the ends and advantages mentioned as well as those inherent therein. While certain preferred embodiments of the disclosure have been illustrated and described for present purposes, numerous changes in the arrangement and construction of parts and steps may be made by those skilled in the art, which changes are encompassed within the scope and spirit of the present disclosure as defined by the appended claims. Each disclosed feature or embodiment may be combined with any of the other disclosed features or embodiments.

What is claimed is:

1. An intraocular lens for implantation in a capsular bag of an eye, the intraocular lens comprising:
a membrane including an anterior face and a posterior face, the anterior and posterior faces joined together at a periphery of the membrane, the membrane including at least one optical element associated therewith;
an internal cavity defined within the membrane;
a gas positioned within the internal cavity, the gas operable to separate the anterior and posterior faces of the membrane; and
wherein the intraocular lens is neutrally buoyant within the capsular bag.

2. The intraocular lens of claim 1, wherein:
the at least one optical element is convex in shape.

3. The intraocular lens of claim 2, wherein:
the gas is positioned adjacent to the posterior face of the membrane.

4. The intraocular lens of claim 3, wherein:
the internal cavity is plano-convex in shape.

5. The intraocular lens of claim 1, wherein:
the at least one optical element includes a first optical element and a second optical element, the at least one optical element being bi-concave in shape.

6. The intraocular lens of claim 5, wherein:
the internal cavity is positioned between the first and second optical elements.

7. The intraocular lens of claim 6, wherein:
the internal cavity is bi-convex in shape.

8. The intraocular lens of claim 6, wherein:
the internal cavity is substantially flat.

9. The intraocular lens of claim 1, wherein:
the membrane includes two or more layers each comprising a different material.

10. The intraocular lens of claim 1, wherein:
the membrane is substantially impervious to the gas positioned within the internal cavity.

11. The intraocular lens of claim 1, wherein:
the gas positioned within the internal cavity is any gas currently approved for medical use.

12. The intraocular lens of claim 1, wherein:
a volume of the at least one optical element is substantially equal to a volume of the gas positioned within the internal cavity.

13. An intraocular lens for implantation in a capsular bag of an eye, the intraocular lens comprising:
a membrane including an anterior face and a posterior face, the anterior and posterior faces joined together at a periphery of the membrane, the membrane including at least one optical element associated therewith; and
an internal cavity defined within the membrane, the internal cavity selectively configurable in an inflated configuration and a deflated configuration, wherein in the inflated configuration the internal cavity includes a gas positioned therein.

14. The intraocular lens of claim 13, wherein:
the at least one optical element is convex in shape;
the internal cavity is positioned adjacent to the posterior face of the membrane; and
the internal cavity is plano-convex in shape.

15. The intraocular lens of claim 13, wherein:
the at least one optical element includes a first optical element and a second optical element, the at least one optical element being bi-concave in shape; and
the internal cavity is positioned between the first and second optical elements.

16. The intraocular lens of claim 15, wherein:
the internal cavity is bi-convex in shape.

17. The intraocular lens of claim 15, wherein:
the internal cavity is substantially flat.

18. The intraocular lens of claim 13, wherein:
the membrane includes two or more materials positioned therein.

19. The intraocular lens of claim 18, wherein:
the two or more materials of the membrane are layered.
